SEQUENCE LISTING (3 sequences)

SEQ ID NO: 1 - protein, 305 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNGLSAWSHPQFEK

SEQ ID NO: 2 - protein, 312 aa, Artificial Sequence
  His-tagged wild-type alpha-hemolysin
MHHHHHHADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNGLSAWSHPQFEK

SEQ ID NO: 3 - protein, 312 aa, Artificial Sequence
  Hexahistidine-tagged alpha-hemolysin with N17C substitution relative to SEQ ID NO 1
MHHHHHHADSDINIKTGTTDIGSCTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTNGLSAWSHPQFEK

(19) United States
(12) Patent Application Publication
(10) Pub. No.: US 2025/0283162 A1
(43) Pub. Date: Sep. 11, 2025

(54) ENGINEERED NANOPORE WITH A NEGATIVELY CHARGED POLYMER THREADED THROUGH THE CHANNEL

(71) Applicant: Roche Sequencing Solutions, Inc., Pleasanton, CA (US)

(72) Inventors: Seong-Ho Shin, Alameda, CA (US); Meng C. Taing, Hayward, CA (US)

(21) Appl. No.: 18/571,034

(22) PCT Filed: Jun. 15, 2022
(86) PCT No.: PCT/EP2022/066284

§ 371 Date: Dec. 15, 2023

Related U.S. Application Data
(60) Provisional application No. 63/202,624, filed on Jun. 17, 2021.

(51) Int. Cl. C12Q 1/6869; C07K 16/00; C12Q 1/48

(52) U.S. Cl. CPC C12Q 1/6869; C07K 16/00; C12Q 1/48

(57) ABSTRACT

Nanopores having charged polymers linked thereto are provided. At least one end of the charged polymer is fixed to or near one end of the channel of the nanopore in a position that enables the charged polymer to enter into the channel. The charged polymer may optionally be fixed in the threaded configuration. When the charged polymer is in the threaded configuration, it enhances the conductivity of the nanopore while still permitting other polymers (such as nucleic acids or polymer tags of tagged nucleotides) to flow through the channel of the nanopore.

### BACKGROUND OF THE INVENTION

### A. Technical Field

Nanopore constructs and their use for nucleic acid sequencing.

### B. Description of Related Art

At their most basic, nanopore sequencing systems comprise a sensing electrode positioned near a nanopore, such that the sensing electrode can detect and record electrochemical characteristics of ions flowing through the nanopore. When relatively large molecules occupy the nanopore, the electrochemical characteristics detected by the sensing electrode change. The identity of the molecule occupying the nanopore can then be determined based upon the change in the electrochemical characteristics, such as a change in current flowing through the nanopore or a decay in measured voltage. An overview of nanopore-based sequencing systems can be found at Wang I and Feng.

The nanopores used in these sequencing systems typically come in one of three flavors: biological nanopores, solid state nanopores, and hybrid nanopores. Biological nanopores are naturally occurring pore-forming molecules, especially proteins such as porins, hemolysins, and the like. Commonly used pore-forming proteins include α-hemolysin (αHL) protein from Staphylococcus aureus, outer membrane protein G (ompG) from Escherichia coli, and porin MspA (MspA) from Mycobacterium smegmatis. In some cases, like in the case of ompG, the pore is formed from a single subunit of the protein. In other cases, like with αHL and MspA, the pore is a multi-subunit assembly of the pore-forming protein. For example, αHL forms a heptameric pore structure and MspA forms an octameric pore structure. Exemplary engineered nanopores based on these proteins can be found at, for example, WO 2016/069806 (αHL), WO 2017/050728 (αHL), WO 2017/184866 (αHL), WO 2018/002125 (αHL), WO 2012/178097 (αHL), Gari (ompG), WO 2017/050722 (ompG), US 2015-0080242 (ompG), Manrao (MspA), Pavlenok (MspA), WO 2013/098562 (MspA), US 2014-0309402 (MspA), US 2013-0146457 (MspA), and Wang II (various). Solid state nanopores are pore structures fabricated from synthetic materials, for example, by forming nanometer-sized holes in synthetic membranes. Exemplary materials from which solid state nanopores can be formed include silicon nitrides, silica, alumina, graphene, boron nitride, and molybdenum disulfide. Solid state nanopores are reviewed by Chen, Lee, Wasfi, Wang I, and Feng. Hybrid nanopores incorporate both biological nanopores and solid state nanopores. For example, a biological nanopore (such as an αHL nanopore) can be inserted into a solid state nanopore. Hybrid nanopores are reviewed by Lee, Wasfi, and Feng.

One approach for nanopore-based nucleic acid sequencing involves threading single stranded nucleic acids directly through the pore (referred to herein as “direct sequencing”). Each nucleotide (or unique combination of nucleotides) generates a unique change in at least one electrochemical characteristic of the pore. These systems frequently use means to control the rate at which the nucleic acid translocates through the pore, such as tethering enzymes to the pore (including polymerases and helicases), removing negatively charged residues from and adding positively charged residues to the pore channel, and adding double stranded regions to the single stranded nucleic acid. Exemplary direct sequencing approaches are discussed by, for example, Feng, Manrao, and Wang I.

Another method involves a sequencing-by-synthesis (SBS) approach by performing a polymerase-catalyzed amplification reaction near an opening of the nanopore with tagged nucleotide polyphosphate molecules. Each tagged nucleotide polyphosphate includes a distinct tag moiety that generates a unique electrochemical signature when it resides in or near the nanopore. As the tagged nucleotide polyphosphates are incorporated into the amplicon, the tag is passed into or near the nanopore, and the electrochemical signature of the tag is recorded. The sequence of the amplicon is derived from the order in which tag moieties enter into the nanopore. Exemplary tag-based SBS approaches and materials for performing such methods are described at, for example, WO 2012-083249, WO 2013/154999, US 2014/0309144, U.S. Pat. No. 9,017,937, WO 2015/148402, WO 2016/069806, WO 2016/144973, US 2016/0222363, US 2016/0333327, WO 2017/050728, WO 2017/184866, WO 2017/050722, US 2017/0267983, US 2018/0245147, US 2018/0094249, WO 2018/002125, and Kumar. Various tags have been proposed for use in such systems, including tags based on polypeptides (such as polylysine tags) and polynucleotides. See, e.g., U.S. Pat. No. 8,652,779 and WO2017042038A1.

### SUMMARY OF THE INVENTION

Disclosed herein are engineered nanopores having a charged polymer threaded through a channel of the nanopore, and their use in nanopore-based sequencing systems and methods. It has been discovered that the inclusion of the charged polymer significantly increases the conductance of the pore, thereby aiding in discriminating different molecules that occupy the pore during a sequencing run (such as groups of nucleotides or polymer tags).

In an embodiment, a charged polymer-linked nanopore (CPL-nanopore) is provided, the CPL-nanopore comprising a channel having an entrance side and an exit side; and a charged polymer threaded through the channel, the charged polymer comprising a negatively charged region disposed in and extending through substantially the entire length of the channel. In some embodiments, a first end of the charged polymer is fixed in place near the entrance side of the channel and a second end of the charged polymer is fixed in place near the exit side of the channel. Exemplary nanopores include those based on α-hemolysin (αHL), outer membrane porin G (OmpG), Mycobacterium smegmatis porin A (MspA), leukocidin nanopore, outer membrane porin F (OmpF) nanopore, cytolysin A (ClyA) nanopore, outer membrane phospholipase A nanopore, Neisseria autotransporter lipoprotein (NalP) nanopore, WZA nanopore, Nocardia farcinica NfpA/NfpB cationic selective channel nanopore, lysenin nanopore, aerolysin, and Curlin sigma S-dependent growth subunit G (CsgG) nanopore. In an exemplary embodiment, the biological nanopore is a heptameric nanopore based on αHL, wherein the heptameric nanopore comprises 7 monomer subunits, each monomer subunit comprising an amino acid sequence having at least 75% sequence identity to SEQ ID NO: 1.

In another embodiment, a system is provided for performing nanopore-based nucleic acid sequencing. The system generally comprises the CPL-nanopores disclosed herein and other elements useful for differentiating molecules occupying the nanopore.

In another embodiment, a method of performing nanopore-based nucleic acid sequencing is provided, using the CPL-nanopore.

Other details and inventions are described in detail herein.

### BRIEF DESCRIPTION OF THE DRAWINGS

FIG. 1A illustrates a charged polymer to be attached to a nanopore.

FIG. 1B illustrates a charged polymer attached at only the first end to a nanopore in an ejected configuration.

FIG. 1C illustrates a charged polymer attached at only the first end to a nanopore in an inserted configuration.

FIG. 1D illustrates a charged polymer attached at the first end near the entrance side of the nanopore channel and at the second end near the exit side of the nanopore in an inserted configuration.

FIG. 1E illustrates a charged polymer attached at the first end near the entrance side of the nanopore channel and at the second end to an external entity near the exit side of the nanopore in an inserted configuration.

FIG. 1F illustrates an exemplary method of fixing a charged polymer to a nanopore in an inserted configuration. The first end is covalently linked to the nanopore and the second end is linked outside of the nanopore.

FIG. 1G illustrates an exemplary method of linking a charged polymer to a nanopore in an inserted configuration. The second end of the charged polymer is covalently linked to the nanopore and the first end is linked outside of the nanopore.

FIG. 2 is a graph of the conductance of a charged polymer-linked nanopore (black squares) versus a nanopore without the charged polymer (grey circles) at various voltage levels.

FIG. 3 is a cross-section of a heptameric alpha-hemolysin nanopore, with the various regions illustrated.

FIG. 4 is a cross-section of a heptameric alpha-hemolysin nanopore having a charged polymer threaded through the channel. The charged polymer is linked to an N17C substitution of the nanopore at a first end and linked to streptavidin via a biotin moiety located at a second end.

FIG. 5 illustrates an exemplary nanopore sequencing complex including a charged polymer-linked nanopore as disclosed herein.

FIG. 6 is a top view of an exemplary nanopore sensor chip including a charged polymer-linked nanopore as disclosed herein.

FIG. 7 illustrates an exemplary nanopore cell comprising a nanopore sequencing complex that includes a charged polymer-linked nanopore as disclosed herein.

FIG. 8 illustrates an exemplary embodiment of an active sequencing complex performing a tag-based SBS nucleic acid sequencing method.

FIG. 9 illustrates an exemplary embodiment of an active sequencing complex performing a direct sequencing method.

FIG. 10 illustrates a specific embodiment of a charged polymer.

FIG. 11 illustrates results of a cation exchange chromatography purification of a 1:6 alpha-hemolysin nanopore having an N17C substitution and a SpyCatcher moiety on one of the monomers.

FIG. 12 is an image of an SDS-PAGE separation of the cation exchange chromatography purification the 1:6 nanopore illustrated in FIG. 11. Lane 0: Molecular weight standards. Lane 1: Peak P1 from cation exchange in the absence of SpyCatcher-GFP. Lane 2: Peak P1 from cation exchange mixed with SpyCatcher-GFP. Lane 3: Peak P2 from cation exchange in the absence of SpyCatcher-GFP. Lane 4: Peak P2 from cation exchange mixed with SpyCatcher GFP.

FIG. 13 shows the results of a series of capture events with a charged polymer-linked alpha-hemolysin nanopore. The charged polymer-linked alpha-hemolysin nanopore was first recorded with the charged polymer only attached to the nanopore at a first end (left of the arrow). Then, streptavidin was flowed onto the chip on the trans side of the barrier (right of the arrow) and recordings were restarted.

### DETAILED DESCRIPTION OF THE INVENTION

The invention will now be described in detail by way of reference only using the following definitions and examples. All patents and publications, including all sequences disclosed within such patents and publications, referred to herein are expressly incorporated by reference.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this invention. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention, which can be had by reference to the specification as a whole. Accordingly, the terms defined immediately below are more fully defined by reference to the specification as a whole.

### I. TERMS

Alpha-hemolysin: As used herein, “alpha-hemolysin,” “α-hemolysin,” and “αHL” are used interchangeably and refer to the monomeric protein that self-assembles into a heptameric water-filled transmembrane channel (i.e., nanopore). Depending on context, the term may also refer to the transmembrane channel formed by seven monomeric proteins.

Base Pair (bp): As used herein, base pair refers to a partnership of adenine (A) with thymine (T), adenine (A) with uracil (U), or of cytosine (C) with guanine (G) in a double stranded nucleic acid.

Capture event: An insertion of a molecule into a nanopore that is sufficient to generate a change in an characteristic of ionic current flowing through the nanopore such that the change is detectable by a sensing electrode.

Complementary: As used herein, the term “complementary” refers to the broad concept of sequence complementarity between regions of two polynucleotide strands or between two nucleotides through base-pairing. It is known that an adenine nucleotide is capable of forming specific hydrogen bonds (“base pairing”) with a nucleotide which is thymine or uracil. Similarly, it is known that a cytosine nucleotide is capable of base pairing with a guanine nucleotide.

Isolated: An “isolated” molecule is a biomolecule that is separated from at least one other molecule with which it is ordinarily associated, for example, in its natural environment.

Monomer subunit: A structural subunit of a multimeric protein complex. For example, a heptameric α-hemolysin pore comprises seven α-hemolysin monomer subunits. A monomer subunit that has not been oligomerized into a multimeric subunit is referred to herein as a “non-oligomerized monomer subunit.”

Monomer unit: A structural subunit of a polymer.

Mutation: As used herein, the term “mutation” refers to a change introduced into a parental sequence, including, but not limited to, substitutions, insertions, and/or deletions (including truncations). The consequences of a mutation include, but are not limited to, the creation of a new character, property, function, phenotype or trait not found in the protein encoded by the parental sequence.

Nanopore: The term “nanopore,” as used herein, generally refers to a pore, channel or passage formed or otherwise provided in an electrically-resistive barrier (such as a lipid membrane, a silicon layer, a polymeric layer, or a graphene layer) through which an ionic current may pass. Unless otherwise stated, the generic term “nanopore” shall include biological nanopores, solid state nanopores, and hybrid nanopores.

Nanopore sequencing complex: A site at which a nanopore-based sequencing method may be performed, generally comprising at least (a) a nanopore configured to establish a current flow through the channel and to permit a molecule of interest (such as a nucleic acid or a polymer tag of a tagged nucleotide) to enter into a channel; and (b) an electrode or set of electrodes configured to detect a characteristic of the nanopore sequencing complex (such as e.g., resistance, capacitance, voltage decay, and ionic current flow).

Native amino acid: Any amino acid of an amino acid sequence that, when aligned with a reference amino acid sequence, is the same as the amino acid occupying the corresponding position of the reference sequence.

Non-native moiety: A component of a nanopore bearing a moiety that is not found in a reference structure. For example, where the nanopore includes a polypeptide, a “non-native amino acid” would be any amino acid having a side chain that represents a substitution or an insertion at a particular position relative to a reference amino acid sequence, or a represents a chemical modification of the side chain of a native amino acid.

Nucleic Acid Molecule: The term “nucleic acid molecule” includes RNA, DNA and cDNA molecules. It will be understood that, as a result of the degeneracy of the genetic code, a multitude of nucleotide sequences encoding a given protein such as α-hemolysin and/or variants thereof may be produced. The present invention contemplates every possible variant nucleotide sequence.

Peptide: The terms “peptide” and “peptide linkage” shall refer to any backbone linkage between two amino acids and/or amino acid analogs resulting from a condensation reaction between a carboxylic acid moiety of one amino acid or amino acid analog and an amino group of a second amino acid or amino acid analog. Unless otherwise clear from the context, these terms shall be understood in all instances as encompassing (but not limited to) linkages between α-amino acids, β-amino acids, γ-amino acids, δ-amino acids, and combinations thereof, as well as linkages between backbone carboxylic acid moieties and side chain amino moieties (such as with ε-linked lysine).

Peptide chain: The term “peptide chain” shall refer to any sequence of two or more amino acids and/or amino acid analogs linked by peptide linkages.

Peptidomimetic: The terms “peptidomimetic” and “peptidomimetic linkage” shall refer to backbone linkages between two amino acid analogs or between an amino acid and an amino acid analog, including but not limited to peptoids (amino acids in which the sidechain is attached to the amino group), azapeptides (replacement of the a-carbon with a nitrogen), oligourea (peptide linkage replaced by a urea linkage), arylamides, oligohydrazides, and the like.

Peptidomimetic chain: The term “peptidomimetic chain” shall refer to any sequence of two or more amino acids and/or amino acid analogs linked by peptidomimetic backbone linkages.

Percent homology: The term “% homology” is used interchangeably herein with the term “% identity” herein and refers to the level of nucleic acid or amino acid sequence identity between the nucleic acid sequence that encodes any one of the inventive polypeptides or the inventive polypeptide's amino acid sequence, when aligned using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for any one of the inventive polypeptides, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is may be used for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. (See, e.g., Altschul, S. F., et al., Nucleic Acids Res. 25:3389-3402, 1997.) Unless stated otherwise, reference to an alignment of two amino acid sequences shall refer an alignment obtainable using the EMBOSS Needle pairwise sequence alignment tool with the BLOSUM62 matrix, GAP OPEN setting of 10, GAP EXTEND setting of 0.5, END GAP PENALTY setting of “false”, END GAP OPEN setting of 10, and END GAP EXTEND setting of 0.5 (available from EMBL-EBI).

Polypeptide: Unless stated otherwise or unless otherwise clear based on the context of the disclosure, the phrase “polypeptide” shall be understood in its broadest sense and shall encompass any sequence of two or more amino acids and/or amino acid analogs linked by peptide linkages and/or peptidomimetic linkages.

Purified: As used herein, “purified” means that a molecule is present in a sample at a concentration of at least 95% by weight, or at least 98% by weight of the sample in which it is contained.

Tag: As used herein, the term “tag” refers to a nanopore-detectable moiety that may be atoms or molecules, or a collection of atoms or molecules. A tag may provide an optical, electrochemical, magnetic, or electrostatic (e.g., inductive, capacitive) signature, which signature may be detected with the aid of a nanopore.

Typically, when a nucleotide is attached to the tag it is called a “Tagged Nucleotide.”

Variant: As used herein, the term “variant” of a reference polypeptide or a nucleic acid is any such molecule that contains at least one molecular change relative to the reference molecule.

### II. CHARGED POLYMERS AND GENERATING THE POLYMER-LINKED NANOPORE

Disclosed herein are biological nanopores having a charged polymer attached thereto in an arrangement that permits the charged polymer to be threaded through a channel of the nanopore.

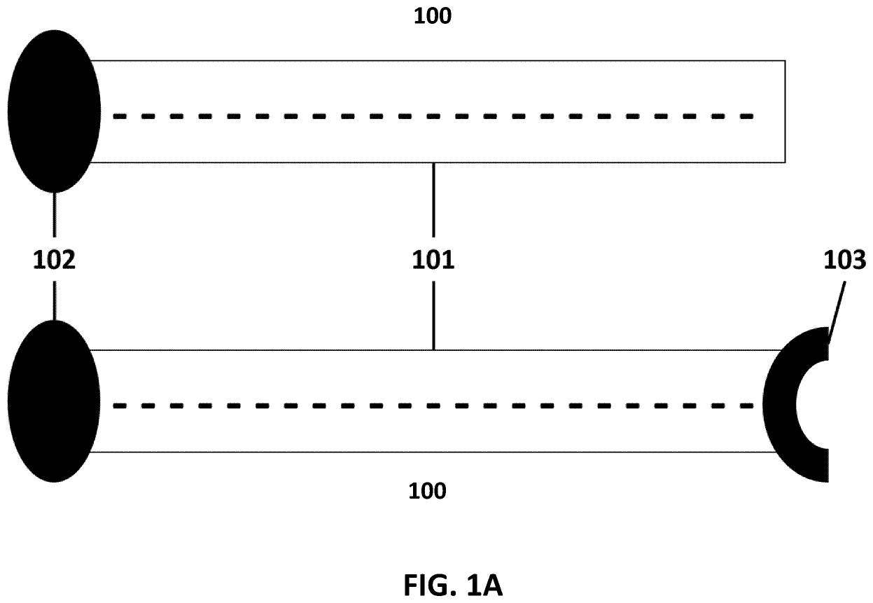

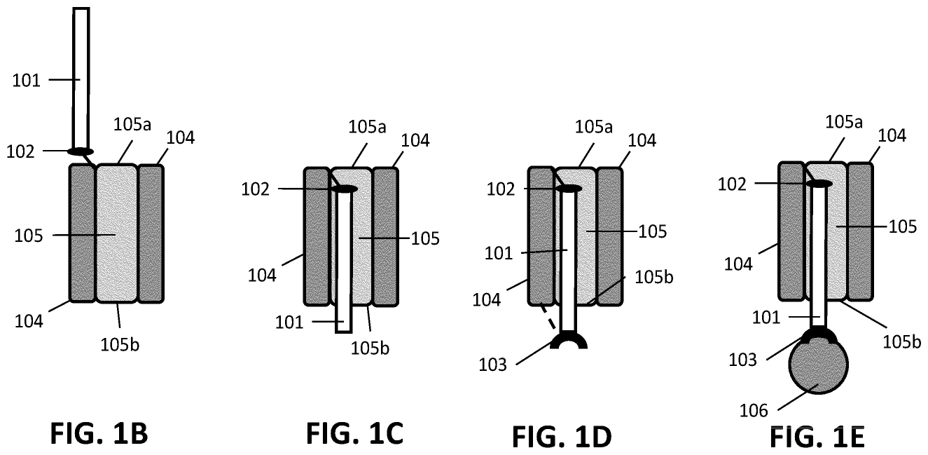

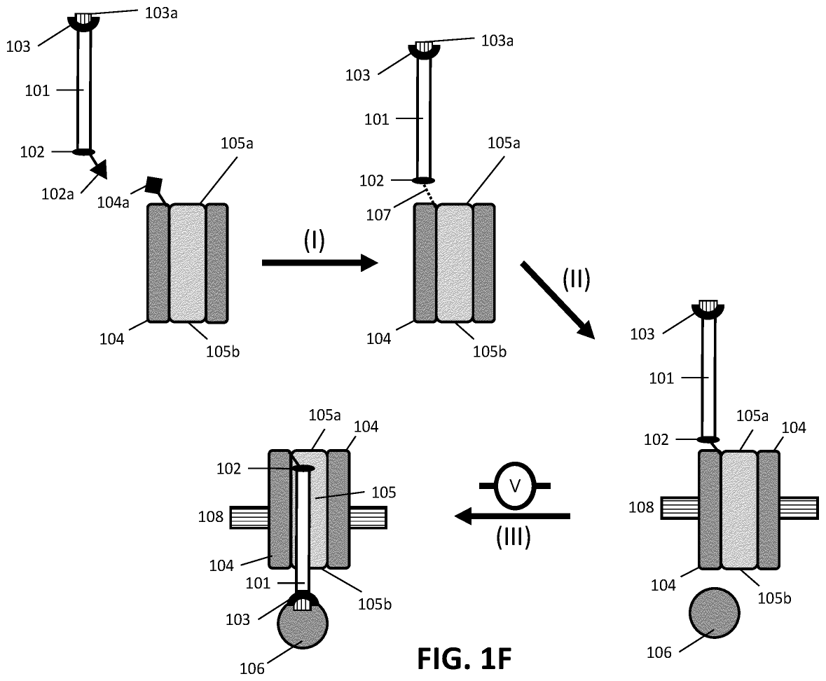

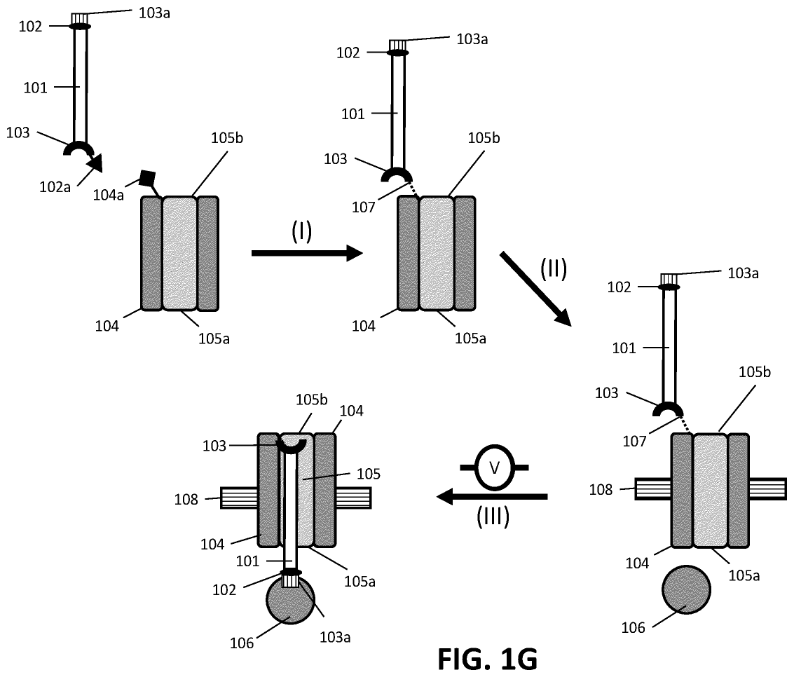

As illustrated in FIG. 1A, the charged polymers 100 comprise at least: (a) a negatively-charged region 101 that has a high density of negative charges; and (2) an entity at or near a first end 102 of the charged polymer 100 that facilitates attachment to the nanopore at or near an entrance to the channel of the nanopore. In some embodiments, the charged polymer 100 further comprises an entity at a second end 103 of the charged polymer 100 that facilitates attachment to either the nanopore or an entity disposed outside of the nanopore. The terms “first end” and “second end” encompass, but are not limited to, attachments formed at the terminal monomeric units of the charged polymer. In some embodiments, the attachments forming the first end 102 can be located at an internal portions of the charged polymer 100, so long as the negatively charged portion 101 can still thread into the channel and increase in conductance of the channel. In some embodiments, the attachments forming the first end 102 and the second end 103 can be located at internal portions of the charged polymer 100, such that the negatively charged portion 101 can still thread into the channel and increase in conductance of the channel and the second end 103 can still be fixed at or near the exit of the channel.

The negatively-charged region 101 comprises a high density of negatively charged monomer units sufficient to enhance the conductivity of the nanopore while still permitting other polymers (such as nucleic acids or polymer tags of tagged nucleotides) to flow through the channel of the nanopore. At least a portion of the monomer units in the negatively charged region possess a negative charge at neutral pH. Moreover, the negatively charged region does not possess a substantial number of monomer units with bulky side chains. Exemplary monomer units include monomer units that comprise phosphate groups capable of forming phosphodiester bonds (such as nucleotides, nucleotide derivatives, and alkyl glycol phosphates (such as ethylene glycol phosphate)), negatively charged polypeptides (such as polypeptides containing high concentrations of aspartic acid or glutamic acid, expressly including polyaspartic acid and polyglutamic acid). In an embodiment, the negatively charged region comprises, consists essentially of, or consists of, a polymer chain of monomer units linked by phosphodiester bonds, including but not limited to nucleotides, nucleotide derivatives, abasic sites (including polymers formed from alkyl glycol phosphates), and combinations thereof. In a further embodiment, the negatively charged region comprises, consists essentially of, or consists of, abasic sites according to the following structure:

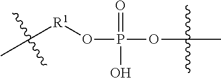

wherein R¹ is an alkyl chain from 2 to 10 carbons in length. In another embodiment, R¹ is 2 carbons in length. In another embodiment, R¹ is 2 carbons in length. In another embodiment, R¹ is 3 carbons in length. In another embodiment, R¹ is 4 carbons in length. In another embodiment, R¹ is 5 carbons in length. In another embodiment, R¹ is 6 carbons in length. In another embodiment, R¹ is 7 carbons in length. In another embodiment, R¹ is 8 carbons in length. In another embodiment, R¹ is 9 carbons in length. In another embodiment, R¹ is 10 carbons in length. In another embodiment, the nanopore the charged polymer has the following structure:

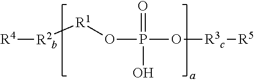

wherein: a is an integer selected such that the length of the charged polymer is at least as long as the length of the channel of the nanopore to which it is connected, R¹ is an alkyl chain from 2 to 10 carbons in length (including all integers between), R² and R³ are nucleotides, b is from 0 to 10, c is from 0 to 10, one of R⁴ and R⁵ is the first end, and the other of R⁴ and R⁵ is the second end. In some embodiments, a is from 10 to 100. In some embodiments, a is from 10 to 90. In some embodiments, a is from 10 to 80. In some embodiments, a is from 10 to 70. In some embodiments, a is from 10 to 60. In some embodiments, a is from 10 to 50. In some embodiments, a is from 20 to 100. In some embodiments, a is from 20 to 90. In some embodiments, a is from 20 to 80. In some embodiments, a is from 20 to 70. In some embodiments, a is from 20 to 60. In some embodiments, a is from 20 to 50.

FIGS. 1B, 1C, 1D, and 1E illustrate cross-sections of a nanopore 104 with a charged polymer attached thereto. The nanopore 104 comprises a channel 105 that runs through the body of the nanopore and forms a path through which molecules can pass from one side of the nanopore to the other. When used for sequencing a nucleic acid or another molecule, the side of the channel through which molecules to be detected enter is termed the channel entrance 105a and the opposite side of the channel is termed the channel exit 105b. The polymer-conjugated pores are formed by fixing the first end 102 in place near to the channel entrance 104. The charged polymer 100 can then be captured by the nanopore, causing the negatively charged portion 102 to thread through the channel and, if desired, beyond the channel exit 105b. In the configuration in which only the first end 102 is fixed in place (FIGS. 1B and 1C), the charged polymer 100 can occupy two configurations: an “ejected” configuration (FIG. 1B), in which the negatively charged portion 101 is substantially outside of the channel 105; and an “inserted” configuration (FIG. 1C) in which the negatively charged portion 101 is threaded through the channel 105 and, if desired, beyond the channel exit 105b. Any method of creating such an attachment may be used. For example, the attachment may be formed by a covalent bond between a reactive moiety on the first end 102 and another reactive moiety on the nanopore or on a surface disposed outside of the nanopore and near the entrance. For example, the first end 102 may comprise N-hydroxysuccinimide (NHS) or Sulfo-NHS, which can be reacted with a primary amine disposed on the nanopore or another surface near the entrance of the pore (such as a bead or a wall of a well in which the nanopore is inserted). As another example, the first end 102 may comprise maleimide, iodoacetyl groups, or pyridyl disulfides, which can be reacted with sulfhydryls (such as cysteine) disposed on the nanopore or another surface near the entrance of the pore (such as a bead or a wall of a well in which the nanopore is inserted). As another example, the first end 102 may comprise a primary amine, which can be reacted (in combination with EDC) with a carboxyl group disposed on the nanopore or another surface near the entrance of the pore (such as a bead or a wall of a well in which the nanopore is inserted). Many other attachment chemistries are known in the art and may be used as well. Alternatively, the first end 102 may comprise a first member of a specific binding pair that interacts with a second member of the specific binding pair that is attached to the nanopore or on a surface disposed outside of the nanopore and near the entrance. Exemplary specific binding pairs include biotin/avidin, biotin/streptavidin, and antibody/epitope. In an embodiment, the first end 102 comprises biotin, wherein the biotin is bound by an avidin or streptavidin tethered to the nanopore or another surface near the entrance of the pore (such as a bead or a wall of a well in which the nanopore is inserted). In another embodiment, the first end 102 comprises an epitope tag (such as a hapten, FLAG tag, HA tag, His tag, Myc tag, V5 tag, Xpress tag, Thrombin tag, BAD tag, Factor Xa tag, VSVG tag, SV40 NLS tag, Protein C tag, S tag, OneStrap tag, or an SB1 tag), wherein the epitope tag is bound by an anti-epitope tag antibody or antibody fragment tethered to the nanopore or another surface near the entrance of the pore (such as a bead or a wall of a well in which the nanopore is inserted).

In some configurations, the charged polymer 100 further comprises a second end 103 that is either fixed at or near the channel exit 105b (FIG. 1D, illustrated by the dashed line between nanopore 104 and second end 103) or outside of the channel exit 105b to a binding entity 106, such as an antibody or a streptavidin molecule (FIG. 1E). When the second end 103 is fixed in place, the charged polymer only occupies the inserted configuration. Any method of creating such an attachment may be used. For example, the second end 103 may be formed by a covalent bond between a reactive moiety of the charged polymer and another reactive moiety on the nanopore or on a surface outside of the nanopore. For example,

N-hydroxysuccinimide (NHS) and Sulfo-NHS are commonly used to covalently attach labels to primary amines; maleimide, iodoacetyl groups and pyridyl disulfides are commonly used to covalently attach labels to sulfhydryls (such as cysteine); primary amines in combination with EDC are commonly used to covalently attach labels to carboxyl groups (such as aspartic acid sidechains, glutamic acid side chains, or the carboxy terminus); and hydrazines and alkoxyamines are commonly used to covalently attach labels to glycoproteins. As another example, the charged polymer 100 may be attached to a first member of a specific binding pair that interacts with a second member of the specific binding pair that is located near the exit side of the channel. Exemplary specific binding pairs include biotin/avidin, biotin/streptavidin, and antibody/epitope. In an embodiment, the second end 103 comprises biotin, wherein the biotin is bound by an avidin or streptavidin tethered to the nanopore near the exit side of the channel or disposed in a well or on a surface outside of the exit side of the channel. In another embodiment, the second end 103 comprises an epitope tag (such as a hapten, FLAG tag, HA tag, His tag, Myc tag, V5 tag, Xpress tag, Thrombin tag, BAD tag, Factor Xa tag, VSVG tag, SV40 NLS tag, Protein C tag, S tag, OneStrap tag, or an SB1 tag), wherein the epitope tag is bound by an anti-epitope tag antibody or antibody fragment tethered to the nanopore near the exit side of the channel or disposed in a well or on a surface outside of the exit side of the channel.

FIG. 1F illustrates an embodiment in which the first end 102 comprises the reactive moiety 102a and the second end 103 comprises the first member of a specific binding pair 103a. The reactive moiety 102a is reacted with a reactive moiety on the nanopore 104a to form a covalent bond 107 between the first end 102 and the nanopore 104 (Arrow I). The reaction products are fractionated to separate the polymer-conjugated nanopores from non-conjugated nanopores by affinity chromatography using the second member of the specific binding pair as a capture agent (Not illustrated). The purified polymer-conjugated nanopores are then inserted into an electrochemically-resistive barrier 108 on a biochip (Arrow II). The channel 105 creates a path for ion flow through the barrier 108, with the channel entrance 105a disposed on a first side of the barrier and the channel exit 105b disposed on a second side of the barrier. The second member of the specific binding pair 106 is disposed on the second side of the barrier in proximity to the channel exit 105b. A potential is established across the barrier (Arrow III), causing the second end 103 to thread through the channel 105 and out the channel exit 105b, where the first member of the specific binding pair 103a interacts with the second member of the specific binding pair 106, thereby fixing the second end 103 in place. This configuration holds the negatively charged portion 101 in the inserted position in the channel.

FIG. 1G illustrates an embodiment in which the first end 102 comprises the first member of the specific binding pair 103a and the second end 103 comprises the reactive moiety 102a. The reactive moiety 102a is reacted with a reactive moiety on the nanopore 104a to form a covalent bond 107 between the second end 103 and the nanopore 104 (Arrow I). The reaction products are fractionated to separate the polymer-conjugated nanopores from non-conjugated nanopores by affinity chromatography using the second member of the specific binding pair as a capture agent (Not illustrated). The purified polymer-conjugated nanopores are then inserted into an electrochemically-resistive barrier 108 on a biochip (Arrow II). The channel 105 creates a path for ion flow through the barrier 108, with the channel entrance 105a disposed on a first side of the barrier and the channel exit 105b disposed on a second side of the barrier. The second member of the specific binding pair 106 is disposed on the first side of the barrier in proximity to the channel entrance 105a. A potential is established across the barrier (Arrow III), causing the first end 102 to thread through the channel 105 and out the channel entrance 105a, where the first member of the specific binding pair 103a interacts with the second member of the specific binding pair 106, thereby fixing the first end 102 in place. This configuration holds the negatively charged portion 101 in the inserted position in the channel.

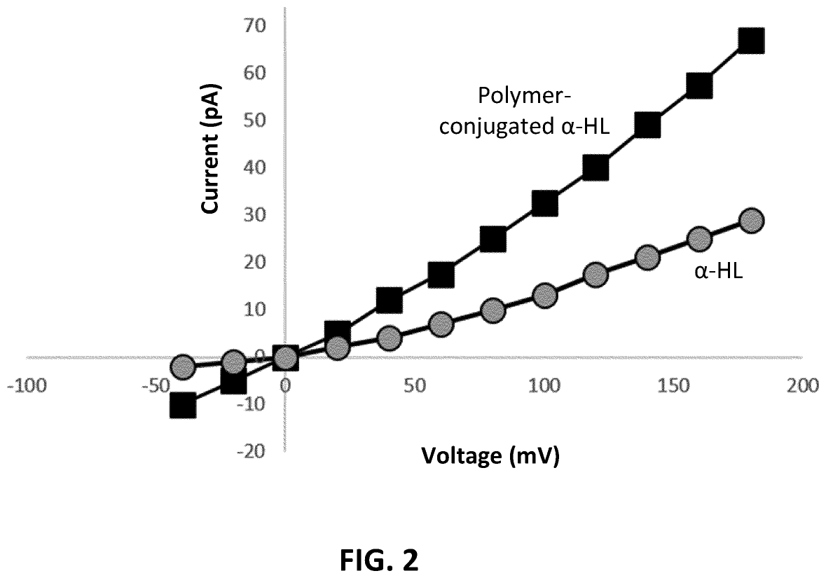

As shown at FIG. 2, when the charged polymer is in the inserted configuration, the conductance of the pore is substantially increased. Without being bound by theory, this enhanced conductance may be due to the polymers carrying large counter-ion clouds, which facilitate the transportation of counter-ions through the channel.

### B. Alpha-Hemolysin Nanopores

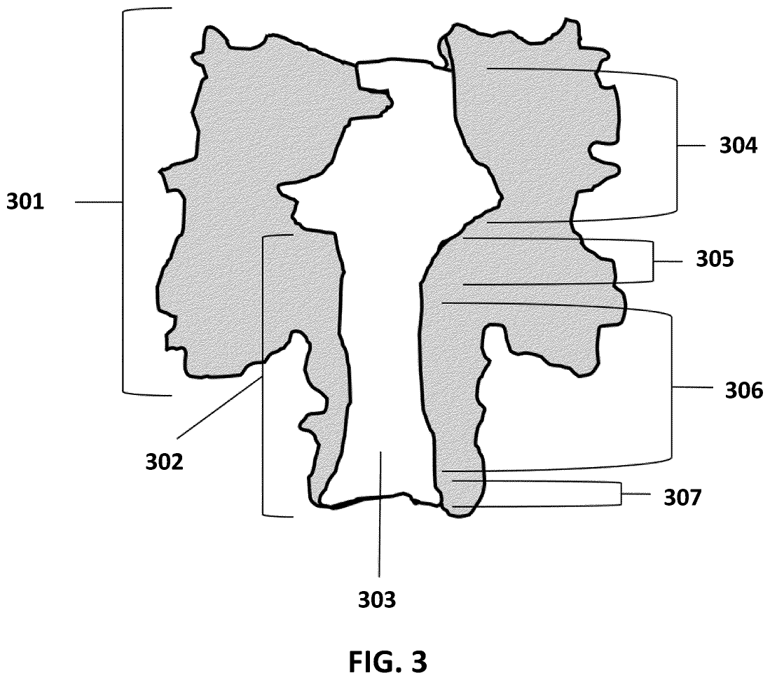

In an embodiment, the nanopore comprises a biological nanopore based on alpha-hemolysin (αHL). αHL nanopores are heptameric structures formed from 7 monomer subunits of the αHL polypeptide from Staphylococcus aureus. A cross-section of an exemplary αHL nanopore is illustrated at FIG. 3. αHL nanopores have a cap region 301 and a beta barrel region 302, with a channel 303 extending axially through the cap and stem regions. The channel 303 can be divided into an entrance 304, a constriction site 305, a beta barrel body 306, and a beta barrel exit 307.

References herein to “beta barrel region” includes each of the constriction site 305, the beta barrel body 306, and the beta barrel exit 307. References herein to “αHL nanopore” shall refer to heptameric pores of 7 αHL monomer subunits. An amino acid sequence corresponding to a wild-type αHL monomer subunit can be found at SEQ ID NO: 1. Unless otherwise indicated, all amino acid numbering relating to αHL monomer subunits are with reference to SEQ ID NO: 1. When reference is made to an αHL monomer subunit “comprising substitution at position #” or “comprising a substitution X #Y” it shall be understood to mean that the monomer subunit amino acid sequence, when aligned with SEQ ID NO: 1, has a substitution at the position corresponding to the recited position of SEQ ID NO: 1. As used herein, a “non-native amino acid” is an amino acid at a position of the monomer subunit amino acid sequence that represents a substitution or insertion when aligned with SEQ ID NO: 1. In an embodiment, the polypeptides comprise at least one αHL monomer subunits having at least 75% identity, at least 80% identity, at least 85% identity, at least 90% identity, at least 91% identity, at least 92% identity, at least 93% identity, at least 94% identity, or at least 95% identity with SEQ ID NO: 1.

Table 1 lists the solvent-facing amino acid residues that are located at the entrance 304, constriction zone 305, or beta barrel 306 when a monomer subunit consisting of SEQ ID NO: 1 is self-assembled into a homoheptameric αHL nanopore in the presence of DPhPC in aqueous solution of 20 mM Tris-HCl pH 8.0, 200 mM NaCl at 37° C. “#” indicates the position within SEQ ID NO: 1, “AA” indicates the amino acid at the recited position of SEQ ID NO: 1, and “Location” indicates the sub-region of the αHL nanopore at which the amino acid is located.

TABLE 1

| # | AA | Location |
|---|---|---|
| 1 | Ala | Entrance |
| 2 | Asp | Entrance |
| 3 | Ser | Entrance |
| 4 | Asp | Entrance |
| 5 | Ile | Entrance |
| 6 | Asn | Entrance |
| 7 | Ile | Entrance |
| 8 | Lys | Entrance |
| 9 | Thr | Entrance |
| 10 | Gly | Entrance |
| 11 | Thr | Entrance |
| 12 | Thr | Entrance |
| 13 | Asp | Entrance |
| 14 | Ile | Entrance |
| 15 | Gly | Entrance |
| 16 | Ser | Entrance |
| 17 | Asn | Entrance |
| 18 | Thr | Entrance |
| 19 | Thr | Entrance |
| 20 | Val | Entrance |
| 21 | Lys | Entrance |
| 22 | Thr | Entrance |
| 46 | Lys | Entrance |
| 47 | Asn | Entrance |
| 106 | Ser | Entrance |
| 111 | Glu | Constriction |
| 113 | Met | Constriction |
| 115 | Thr | Beta Barrel |
| 117 | Thr | Beta Barrel |
| 119 | Gly | Beta Barrel |
| 121 | Asn | Beta Barrel |
| 123 | Asn | Beta Barrel |
| 125 | Thr | Beta Barrel |
| 129 | Thr | Exit |
| 130 | Gly | Exit |
| 131 | Lys | Exit |
| 133 | Gly | Beta Barrel |
| 135 | Leu | Beta Barrel |
| 137 | Gly | Beta Barrel |
| 139 | Asn | Beta Barrel |
| 141 | Ser | Beta Barrel |
| 143 | Gly | Beta Barrel |
| 145 | Thr | Beta Barrel |
| 147 | Lys | Constriction |
| 149 | Val | Entrance |
| 239 | Ser | Entrance |

These sites can generally be modified (such as by substitutions, insertions, or deletions) to modify various characteristics of the nanopore. Exemplary engineered αHL nanopores useful with the present invention can be found at, for example, Ayub, Wang II, WO 2014/100481, WO 2016/069806, WO 2017/050718, WO 2017/184866, and WO 2018/002125 (each of which is incorporated by reference).

The αHL monomer subunits of the nanopore may comprise modifications that confer specific characteristics on the pore. One example includes substitutions that control the ability of non-oligomerized monomer subunits to self-oligomerize. For example, αHL monomer subunits having substitutions at H35 (e.g., H35G/L/D/E substitutions) are substantially non-oligomerized as long as they are kept at room temperature or below (e.g. 25° C. or lower), but will stably oligomerize when the temperature is raised to a higher temperature (e.g. 35° C.). In an exemplary embodiment, the αHL monomer subunits further comprise an H35G/L/D/E substitution. Other examples of substitution strategies for controlling self-oligomerization and/or directing specific patterns of oligomerization are disclosed at, for example, WO/2017/050718. Another example includes substitutions that improve the expression level of the αHL monomer subunit(s) in a recombinant cell used to express the monomer subunit(s). Other examples include substitutions that reduce coefficient of variation of the arrival rate of the pore (CV), such as D227N.

In some embodiments, the nanopore is a narrow pore. Reference herein to a “narrow” pore shall mean that at least 6 monomer subunits of the pore comprise E111, M113, and K147 or the nanopore comprises substitutions at E111, M113, and/or K147 sufficient to narrow the constriction site relative to a nanopore having 6 monomer subunits of the pore comprises E111, M113, and K147. In other embodiments, the nanopore includes substitutions that widen the constriction site. These substitutions replace the sidechain of the amino acids forming the constriction site with amino acids having shorter and/or less bulky side chains. Examples include E111A/S, M113A/S, and K147A/S/N substitutions. In an example, at least 3 monomer subunits of the αHL nanopore comprise one or more substitutions selected from the group consisting of E111A/S, M113A/S, and K147A/S/N. In an example, at least 4 monomer subunits of the αHL nanopore comprise one or more substitutions selected from the group consisting of E111A/S, M113A/S, and K147A/S/N. In an example, at least 5 monomer subunits of the αHL nanopore comprise one or more substitutions selected from the group consisting of E111A/S, M113A/S, and K147A/S/N. In an example, at least 6 monomer subunits of the αHL nanopore comprises one or more substitutions selected from the group consisting of E111A/S, M113A/S, and K147A/S/N (including 6:1 monomer subunits, wherein the “6” component has substitutions corresponding to E111A/S, M113A/S, and K147A/S/N).

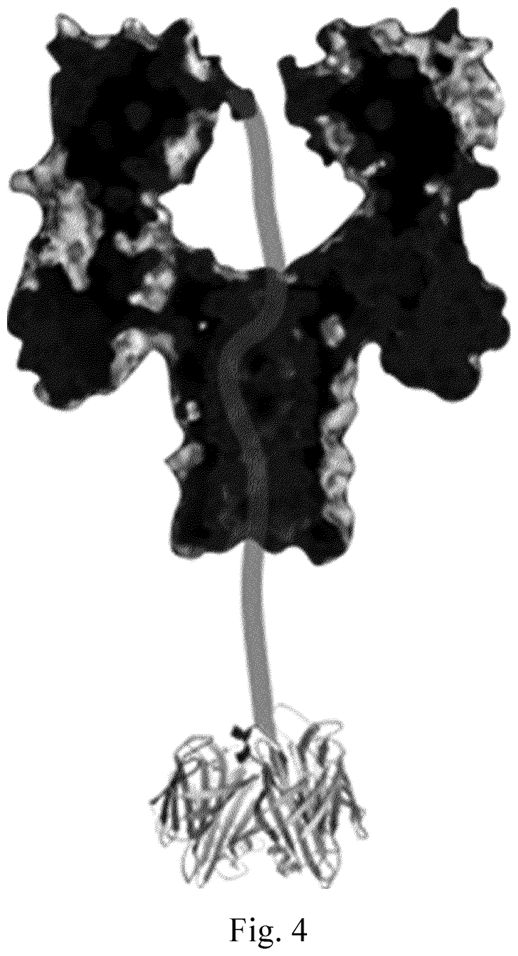

Each monomer subunit of the αHL nanopore may have the same primary amino acid sequence (termed a “homoheptamer”), or at least one monomer subunit of the heptamer may have an amino acid sequence that is different from the amino acid sequence of the other monomer subunits (termed a “heteroheptamer”). Heteroheptameric αHL nanopores may be referred to herein by a ratio of the species of different monomer subunits used in the nanopore. For example, a “6:1 αHL nanopore” has 6 monomer subunits with the same amino acid sequence and 1 monomer subunit with a different amino acid sequence. In such an example, reference to the “6” component shall mean each of the 6 identical monomer subunits, while reference to the “1” component shall mean the 1 monomer subunit with the different amino acid sequence. In some embodiments, each monomer subunit of the αHL nanopore is disposed in a polypeptide that does not contain additional monomer subunits (termed herein a “non-oligomerized monomer subunit”). Exemplary methods of making homoheptamers and heteroheptamers from non-oligomerized monomer subunits are disclosed at US 2017-0088890 A1. For example, 6:1 heteroheptamers can be generated by mixing two different monomer preparations (for example, one in which the monomer is modified with an entity that can be used to bind to a polymerase and another entity that does not contain such a modification). The entity that is intended to be in excess in the resulting heptamer is provided in a molar excess relative to the other heptamer in the presence of a membrane and the mixture is incubated in an aqueous solution (such as 20 mM Tris-HCl pH 8.0, 200 mM NaCl or 20 mM Sodium Citrate pH 3, 400 mM NaCl, 0.1% TWEEN20+0.2 M TMAO) overnight at 37° C. The resulting heptamers are then purified by cation exchange chromatography. In some embodiments, oligomerization is performed in the presence of trimethylamine N-oxide (TMAO), such as from 0.1 to 5M TMAO, from 1 to 4M TMAO, and the like. In an embodiment, an αHL monomer subunit having a set of substitutions relative to SEQ ID NO: 1 comprising an H35G substitution is oligomerized in the presence of an aqueous buffer comprising from 0.1 to 5M TMAO at 37° C. In another embodiment, an αHL monomer subunit having a set of substitutions relative to SEQ ID NO: 1 comprising an H35G substitution is oligomerized in the presence of an aqueous buffer comprising from 0.2 to 4M TMAO at 37° C. In another embodiment, an αHL monomer subunit having a set of substitutions relative to SEQ ID NO: 1 comprising an H35G substitution is oligomerized in the presence of an aqueous buffer comprising about 0.2M to about 3M TMAO at 37° C. In other embodiments, the nanopore includes at least one set of concatenated monomer subunits. Exemplary methods of making αHL nanopores from concatenated monomer subunits of αHL monomer subunits are disclosed at, for example, Hammerstein and US 2017-0088890 A1. In some embodiments, the αHL nanopore is a 6:1 nanopore, wherein the charged polymer is attached to the “1” component. In a further embodiment, the “1” component comprises an N17C substitution to facilitate attachment of the charged polymer to the “1” subunit. In yet another embodiment, the “1” subunit comprises the N17C substitution and either the first end or the second end of the charged polymer comprises a maleimide to facilitate attachment of the charged monomer to the “1” subunit. In yet another embodiment, the “1” subunit comprises the N17C substitution and either the first end or the second end of the charged polymer comprises a maleimide to facilitate attachment of the charged monomer to the “1” subunit and the other of the first and second ends comprises a first member of a specific binding pair (such as biotin or an epitope tag). FIG. 4 illustrates an embodiment comprising 1 αHL monomer subunit having a N17C substitution, wherein the charged polymer is attached to the N17C via reaction of the cysteine sidechain with a maleimide moiety disposed at the first end of the charged polymer and further wherein the second end is fixed in place outside of the exit side of the channel via attachment between a biotin molecule disposed on the second end of the charged polymer and a streptavidin bead.

The αHL nanopores described herein may also include a polymerase attached thereto. Such an embodiment is especially useful for performing tag-base SBS methods. In an embodiment, a single polymerase is attached to the αHL nanopore. Exemplary polymerases include those derived from DNA polymerase Clostridium phage phiCPV4 (described by GenBank Accession No. YP_00648862, referred to herein as “Pol6”), phi29 DNA polymerase, T7 DNA pol, T4 DNA pol, E. coli DNA pol 1, Klenow fragment, T7 RNA polymerase, and E. coli RNA polymerase, as well as associated subunits and cofactors. In an embodiment, the polymerase is a DNA polymerase derived from Pol6. Exemplary Pol6 derivatives useful in nanopore-based sequencing are disclosed at, for example, US 2016/0222363, US 2016/0333327, US 2017/0267983, US 2018/0094249, and US 2018/0245147. Exemplary methods of attaching a polymerase to an αHL nanopore include Spy Tag/SpyCatcher peptide system (Zakeri et al. PNAS 109: E690-E697 2012), native chemical ligation system (Thapa et al., Molecules 19:14461-14483 2014), sortase system (Wu and Guo, J Carbohydr Chem 31:48-66 2012; Heck et al., Appl Microbiol Biotechnol 97:461-475 2013)), transglutaminase systems (Dennler et al., Bioconjug Chem 25:569 578 2014), formylglycine linkage systems (Rashidian et al., Bio conjug Chem 24:1277-1294 2013), Click chemistry attachment systems, or other chemical ligation techniques known in the art. In yet other embodiments, one of the αHL monomer subunits is expressed as a fusion protein with the polymerase. In an embodiment, the polymerase is attached to an amino acid side chain of one of the monomer subunits. In an embodiment, the αHL nanopore is a 5:1:1 nanopore, wherein the polymerase is attached to one of the “1” components and the charged polymer is attached to the other “1” component. In a specific embodiment, a 5:1:1 nanopore is provided, wherein one of the “1” components comprises a member of a SpyCatcher/Spy Tag attachment system and the other “1” component comprises an N17C substitution. In another embodiment, the αHL nanopore is a 6:1 nanopore, wherein the polymerase and the charged polymer are attached to the “1” component, and wherein the polymerase is a DNA polymerase.

### C. Alternative Nanopores

In an embodiment, the nanopore comprises a biological nanopore that is not αHL. Exemplary non-αHL biological nanopores include outer membrane porin G (OmpG) nanopore from Escherichia coli (canonical full-length unprocessed sequence disclosed at Uniprot Accession No. P76045-1), Mycobacterium smegmatis porin A (MspA) (canonical full-length unprocessed sequence disclosed at Uniprot Accession No. AOQR29-1, dodecameric connector channel from bacteriophage phi29 DNA packaging motor (Phi29), Bacillus anthracis protective antigen, PA63 (PA63), leukocidin nanopore, outer membrane porin F (OmpF) nanopore, ferric hydroxamate uptake component A (FhuA) from E. coli, cytolysin A (ClyA) nanopore, outer membrane phospholipase A nanopore, Neisseria autotransporter lipoprotein (NalP) nanopore, WZA nanopore, Nocardia farcinica NfpA/NfpB cationic selective channel nanopore, lysenin nanopore, aerolysin, DNA packaging motor of bacteriophage SPP1 (SPP1), and Curlin sigma S-dependent growth subunit G (CsgG) nanopore. Reviews of the use of various nanopore proteins can be found at, for example, Gari (OmpG), Haque et al. (MspA and Phi29), and Wang II (Phi29, MspA, CsgG, PA63, ClyA, FhuA, SPP1).

### III. NUCLEIC ACID SEQUENCING SYSTEMS AND METHODS

Systems and methods for performing nucleic acid sequencing using the disclosed nanopores are provided. Systems for nanopore-based nucleic acid sequencing generally comprise a chip with a plurality of nanopore sequencing complexes that include the charged polymer-linked nanopores as disclosed herein and a computing system adapted to record changes in one or more electrical characteristics of the nanopore sequencing complexes.

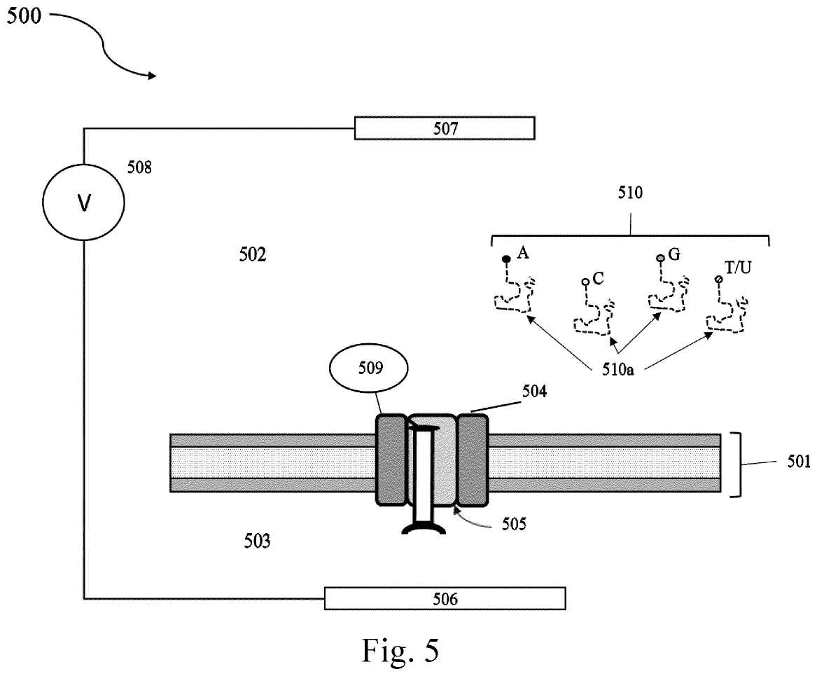

FIG. 5 illustrates an exemplary nanopore sequencing complex 500. An electrochemically resistive barrier 501 separates a first electrolyte solution 502 from a second electrolyte solution 503. The side of the barrier on which the first electrolyte solution is disposed is termed the cis side of the barrier, which the side on which the second electrolyte solution is disposed is termed the trans side. A nanopore having the charged polymer attached thereto as described herein 504 is inserted into the barrier 501, wherein the entrance side of the channel and the first end of the charged polymer are oriented toward the cis side of the barrier and the exit side of the channel and the second end of the charged polymer are oriented toward the trans side of the barrier, such that the channel 505 permits ion exchange between the first electrolyte solution and the second electrolyte solution. A working electrode 506 and a counter electrode 507 are operatively coupled to a signal source 508. The signal source 508 applies a voltage signal between the working electrode 506 and the counter electrode 507. The nanopore 504 is positioned with respect to the electrodes such that changes in at least one electrical characteristic of the nanopore can be detected and transmitted to the computing system. Where the system is used for sequencing-by-synthesis methods, the system further comprises (f) a nucleic acid polymerase 507 associated with the nanopore on the cis side of the barrier; and (g) a set of polymer tagged nucleoside-5′-oligophosphates (N5OP) 510 disposed in the first electrolyte solution.

Any semi-permeable membrane that permits the transmembrane flow of water but has limited to no permeability to the flow of ions or other osmolytes may be used as an electrochemically-resistive barrier, so long as the nanopores described herein can be inserted. For example, the disclosed methods and systems can be used with membranes that are polymeric. In some embodiments, the membrane is a copolymer. In some embodiments, the membrane is a triblock copolymer. In an exemplary embodiment, the membrane is an A-B-A triblock copolymer wherein “A” is poly-b-(methyloxazoline) and “B” is poly(dimethylsiloxane)-poly-b-(methyloxazoline) (Pmoxa-PDMS-Pmoxa membrane). In other embodiments, the electrochemically-resistive barrier may be a lipid bilayer. Exemplary materials used to form lipid bilayers include, for example, phospholipids, for example, selected from diphytanoyl-phosphatidylcholine (DPhPC), 1,2-diphytanoyl-sn-glycero-3-phosphocholine, 1,2-di-O-phytanyl-sn-glycero-3-phosphocholine (DOPhPC), palmitoyl-oleoyl-phosphatidylcholine (POPC), dioleoyl-phosphatidyl-methylester (DOPME), dipalmitoylphosphatidylcholine (DPPC), phosphatidylcholine, phosphatidylethanolamine, phosphatidylserine, phosphatidic acid, phosphatidylinositol, phosphatidylglycerol, sphingomyelin, 1,2-di-O-phytanyl-sn-glycerol, 1,2-dipalmitoyl-sn-glycero-3-phosphoethanolamine-N-[methoxy (polyethylene glycol)-350], 1,2-dipalmitoyl-sn-glycero-3-phosphoethanolamine-N-[methoxy (polyethylene glycol)-550], 1,2-dipalmitoyl-sn-glycero-3-phosphoethanolamine-N-[methoxy (polyethylene glycol)-750], 1,2-dipalmitoyl-sn-glycero-3-phosphoethanolamine-N-[methoxy (polyethylene glycol)-1000], 1,2-dipalmitoyl-sn-glycero-3-phosphoethanolamine-N-[methoxy (polyethylene glycol)-7000], 1,2-dioleoyl-sn-glycero-3-phosphoethanolamine-N-lactosyl, GM1 Ganglioside, Lysophosphatidylcholine (LPC), or any combination thereof.

The electrochemically-resistive barrier 501 separates the second electrolyte solution 503 on the trans side of the barrier from the first electrolyte solution 502 on the cis side of the barrier. The first electrolyte 502 and second electrolyte 503 are aqueous solutions buffered to an optimum ion concentration and maintained at an optimum pH to keep the nanopore open and the barrier intact as long as possible. The first electrolyte solution can comprise free nanopores (prior to insertion in the barrier), the nucleic acid of interest, and any ancillary reagents needed to sequence the nucleic acid of interest (such as primer nucleic acids and N5OPs for SBS sequencing methods). The first and second electrolyte solutions may further comprise one or more of the following: lithium chloride (LiCl), sodium chloride (NaCl), potassium chloride (KCl), lithium glutamate, sodium glutamate, potassium glutamate, lithium acetate, sodium acetate, potassium acetate, calcium chloride (CaCl₂)), strontium chloride (SrCl₂), manganese chloride (MnCl₂), and magnesium chloride (MgCl₂).

A single free nanopore (not illustrated) can be inserted into barrier 501 by an electroporation process caused by the voltage signal, thereby forming a nanopore 504 in barrier 501. The channel 505 crosses the barrier 501 and provides the only path for ionic flow from the first electrolyte 502 to working electrode 506.

In some embodiments, working electrode 506 is a metal electrode. For non-faradaic conduction, working electrode 506 can be made of metals or other materials that are resistant to corrosion and oxidation, such as, for example, platinum, gold, titanium nitride, and graphite. For example, working electrode 506 can be a platinum electrode with electroplated platinum. In another example, working electrode 506 can be a titanium nitride (TiN) working electrode. Working electrode 506 can be porous, thereby increasing its surface area and a resulting capacitance associated with working electrode 506. Because the working electrode of a nanopore sequencing complex can be independent from the working electrode of another nanopore sequencing complex, the working electrode can be referred to as cell electrode in this disclosure.

Counter electrode (CE) 507 can be an electrochemical potential sensor. In some embodiments, counter electrode 507 is shared between a plurality of nanopore sequencing complexes, and can therefore be referred to as a common electrode. The common electrode can be configured to apply a common potential to the first electrolyte 502 in contact with the nanopore 504. Counter electrode 507 and working electrode 506 can be coupled to signal source 508 for providing electrical stimulus (e.g., voltage bias) across barrier 501, and can be used for sensing electrical characteristics of barrier 501 (e.g., resistance, capacitance, voltage decay, and ionic current flow). A signal source 508 can apply a voltage signal between working electrode 506 and counter electrode 507.

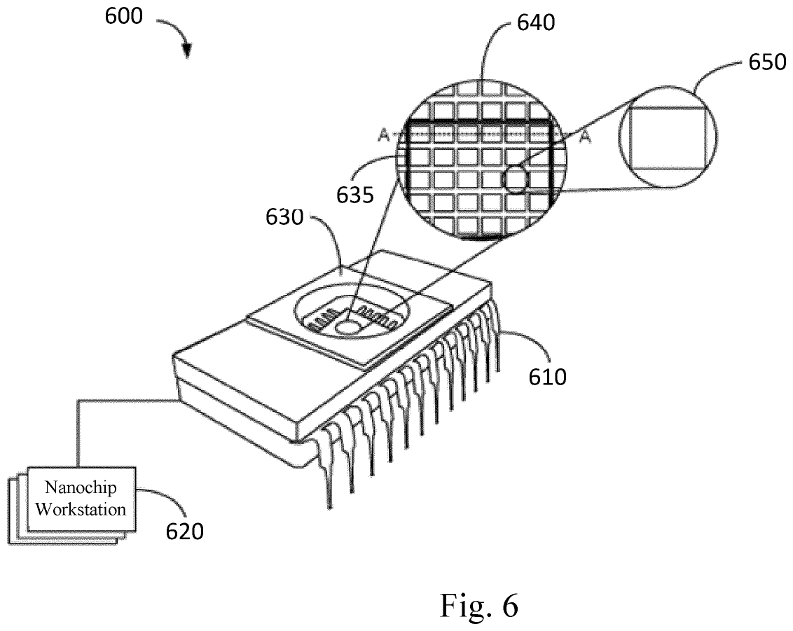

FIG. 6 is a top view of an exemplary embodiment of a nanopore sensor chip 600 having an array 640 of nanopore cells 650, each nanopore cell comprising a single nanopore sequencing complex 500. Each nanopore cell 650 may include a control circuit integrated on a silicon substrate of nanopore sensor chip 600. In some embodiments, side walls 636 are included in array 640 to separate groups of nanopore cells 650 so that each group can receive a different sample for characterization. Each nanopore cell can be used to sequence a nucleic acid. In some embodiments, nanopore sensor chip 600 includes a cover plate 630. In some embodiments, nanopore sensor chip 600 also includes a plurality of pins 610 for interfacing with other circuits, such as a computer processor.

In some embodiments, nanopore sensor chip 600 includes multiple chips in a same package, such as, for example, a Multi-Chip Module (MCM) or System-in-Package (SiP). The chips can include, for example, a memory, a processor, a field-programmable gate array (FPGA), an application-specific integrated circuit (ASIC), data converters, a high-speed I/O interface, etc.

In some embodiments, nanopore sensor chip 600 is coupled to (e.g., docked to) a nanochip workstation 620, which can include various components for carrying out (e.g., automatically carrying out) various embodiments of the processes disclosed herein. These process can include, for example, analyte delivery mechanisms, such as pipettes for delivering lipid suspension or other membrane structure suspension, analyte solution, and/or other liquids, suspension or solids. The nanochip workstation components can further include robotic arms, one or more computer processors, and/or memory. A plurality of polynucleotides can be detected on array 640 of nanopore cells 650. In some embodiments, each nanopore cell 650 is individually addressable.

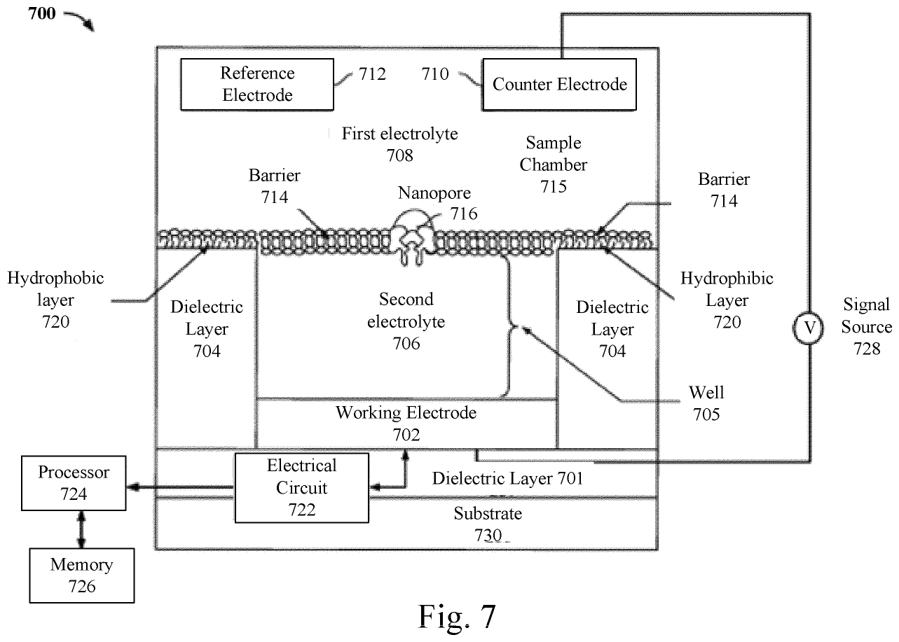

FIG. 7 illustrates an exemplary embodiment of a nanopore cell comprising a nanopore sequencing complex. Nanopore cell 700 can include a well 705 formed of dielectric layers 701 and 704; the barrier 714 formed over well 705; and a sample chamber 715 separated from well 705 by the barrier 714. Well 705 can contain a volume of the second electrolyte 706, and the sample chamber 715 can hold the first electrolyte 708 containing a nanopore, and the analyte of interest (e.g., a nucleic acid molecule to be sequenced). Nanopore cell 700 can include a working electrode 702 at the bottom of well 705 and a counter electrode 710 disposed in sample chamber 715. A signal source 728 can apply a voltage signal between working electrode 702 and counter electrode 710. A single nanopore can be inserted into barrier 714 by an electroporation process caused by the voltage signal, thereby forming a nanopore 716 in the barrier 714. The barrier (e.g., lipid bilayers 714 or other membrane structures) in the array can be neither chemically nor electrically connected to each other. Thus, each nanopore cell in the array can be an independent sequencing machine, producing data unique to the single polymer molecule associated with the nanopore that operates on the analyte of interest and modulates the ionic current through the otherwise impermeable barrier.

As shown in FIG. 7, nanopore cell 700 can be formed on a substrate 730, such as a silicon substrate. Dielectric layer 701 can be formed on substrate 730. Dielectric material used to form dielectric layer 701 can include, for example, glass, oxides, nitrides, and the like. An electric circuit 722 for controlling electrical stimulation and for processing the signal detected from nanopore cell 700 can be formed on substrate 730 and/or within dielectric layer 701. For example, a plurality of patterned metal layers (e.g., metal 1 to metal 6) can be formed in dielectric layer 701, and a plurality of active devices (e.g., transistors) can be fabricated on substrate 730. In some embodiments, signal source 728 is included as a part of electric circuit 722. Electric circuit 722 can include, for example, amplifiers, integrators, analog-to-digital converters, noise filters, feedback control logic, and/or various other components. Electric circuit 722 can be further coupled to a processor 724 that is coupled to a memory 726, where processor 724 can analyze the sequencing data to determine sequences of the polymer molecules that have been sequenced in the array.

Working electrode 702 can be formed on dielectric layer 701, and can form at least a part of the bottom of well 705.

Dielectric layer 704 can be formed above dielectric layer 701. Dielectric layer 704 forms the walls surrounding well 705. Dielectric material used to form dielectric layer 704 can include, for example, glass, oxide, silicon mononitride (SiN), polyimide, or other suitable hydrophobic insulating material. The top surface of dielectric layer 704 can be silanized. The silanization can form a hydrophobic layer 720 above the top surface of dielectric layer 704. In some embodiments, hydrophobic layer 720 has a thickness of about 1.5 nanometer (nm).

Well 705 formed by the dielectric layer walls 704 includes a second electrolyte 706 in contact with the working electrode 702. In some embodiments, second electrolyte 706 has a thickness of about three microns (μm).

The barrier 714 is formed on top of dielectric layer 704 and spanning across well 705. Barrier 714 is embedded with a single nanopore having a charged polymer attached thereto as disclosed herein 716. Nanopore 716 can be large enough for passing at least a portion of the analyte of interest, the charged polymer, and/or small ions (e.g., Na⁺, K⁺, Ca²⁺, Cl⁻) between the two sides of barrier 714. Sample chamber 715 is disposed on the cis side of barrier 714, and can hold a solution of the analyte of interest for characterization.

In some embodiments, various checks are made during creation of the nanopore cell as part of calibration. Once a nanopore cell is created, further calibration steps can be performed, e.g., to identify nanopore cells that are performing as desired (e.g., one nanopore in the cell). Such calibration checks can include physical checks, voltage calibration, open channel calibration, and identification of cells with a single nanopore.

In use, an active sequencing complex is generated at a plurality of nanopore sequencing complexes, a molecule enters into the channel of the nanopore to cause a change in one or more electrical characteristics of the nanopore sequencing complex, the changes are detected and transmitted to the computing system, and the computing system correlates. In a SBS sequencing method, the molecule that enters the channel is a polymer tag of a tagged N5OP. In direct sequencing methods, the molecule that enters the channel is the nucleic acid of interest.

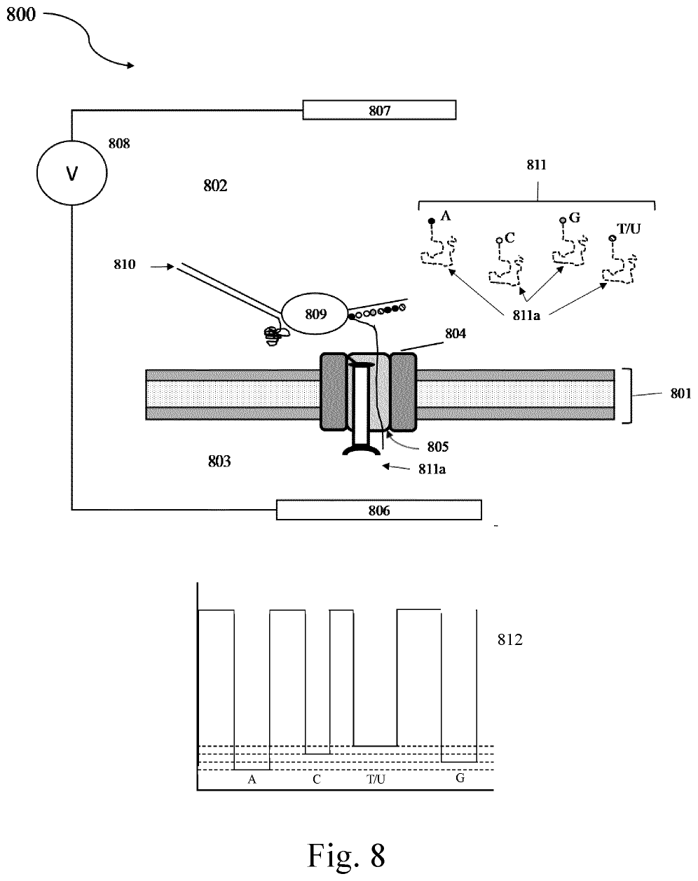

FIG. 8 illustrates an exemplary embodiment of an active sequencing complex 800 for performing a tag-based SBS nucleic acid sequencing. The electrically-resistive barrier 801 separates the first electrolyte solution 802 from the second electrolyte solution 803. The nanopore 804 is disposed in the electrically-resistive barrier 801, and the channel of the nanopore 805 provides a path through which ions can flow between the first electrolyte 802 and the second electrolyte 803. The working electrode 806 is disposed on the side of the electrically-resistive barrier 801 containing the second electrolyte 803 (termed the “trans side” of the electrically-resistive barrier) and positioned near the nanopore 804. The counter electrode 807 is positioned on the side of the electrically-resistive barrier 801 containing the first electrolyte 802 (termed the “cis side” of the electrically-resistive barrier). The signal source 808 is adapted to apply a voltage signal between the working electrode 806 and the counter electrode 807. A polymerase 809 is associated with nanopore 804, and a primed template nucleic acid 810 is associated with the polymerase 809. The first electrolyte 802 includes four different polymer-tagged nucleoside oligophosphates 811 (tag illustrated as 811a). The polymerase 809 catalyzes incorporation of the polymer-tagged nucleotides 811 into an amplicon of the template. When a polymer-tagged nucleoside oligophosphate 811 is correctly complexed with polymerase 809, the tag 811a can be pulled (e.g., loaded) into the nanopore by an electrical force, such as a force generated in the presence of an electric field generated by a voltage applied across the electrically-resistive barrier 801 and/or nanopore 804. While the tag 811a occupies the channel of the nanopore 804, it affects ionic flow through the nanopore 804, thereby generating an ionic blockade signal 812. Each nucleotide 811 has a unique polymer tag 811a that generates a unique ionic blockade signal due to the distinct chemical structure and/or size of the tag 811a. By identifying the unique ionic blockade signal 812, the identity of the unique tags 811a (and therefore, the nucleotide 810 with which it is associated) can be identified. This process is repeated iteratively with each nucleotide 811 incorporated into the amplicon. Exemplary tag-based SBS approaches and materials for performing such methods are described at, for example, WO 2012-083249, WO 2013/154999, US 2014/0309144, U.S. Pat. No. 9,017,937, WO 2015/148402, WO 2016/069806, WO 2016/144973, US 2016/0222363, US 2016/0333327, WO 2017/050728, WO 2017/184866, WO 2017/050722, US 2017/0267983, US 2018/0245147, US 2018/0094249, WO 2018/002125, and Kumar (each of which is incorporated herein by reference). Various tags have been proposed for use in such systems, including tags based on polypeptides (such as polylysine tags), polynucleotides, and polyethylene glycol. See, e.g., U.S. Pat. No. 8,652,779 and WO2017042038A1 (each of which is incorporated herein by reference).

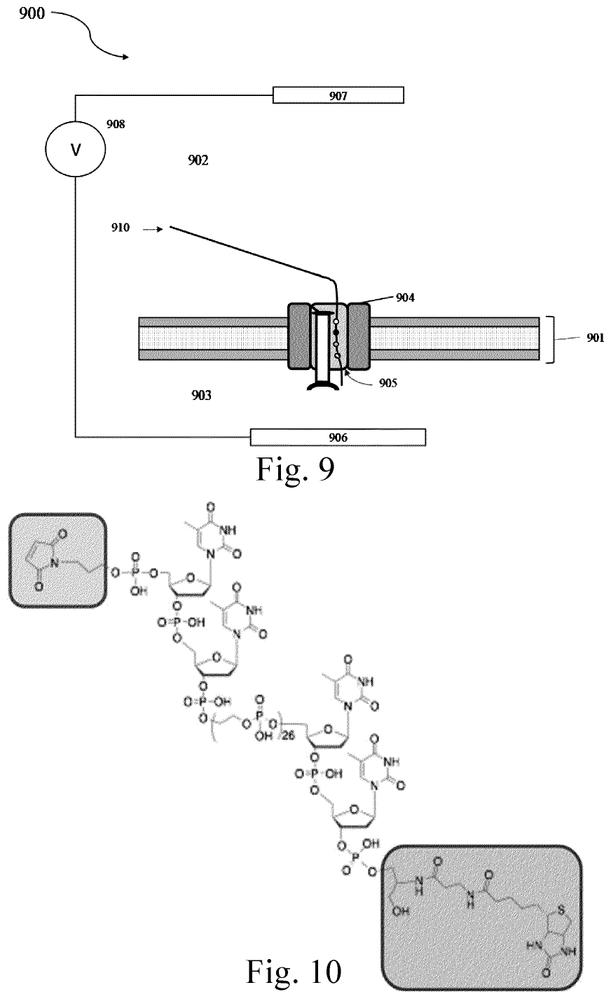

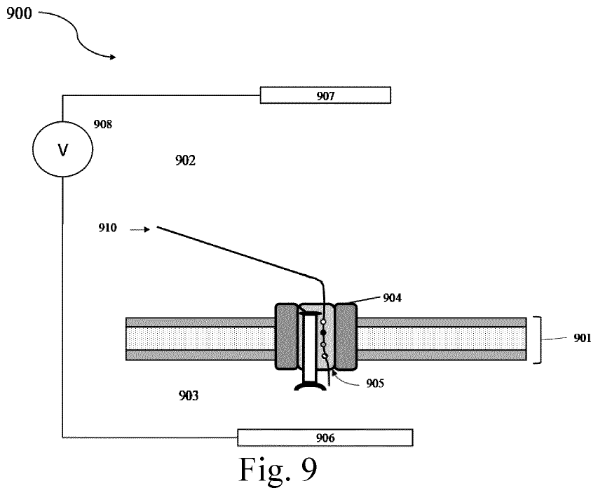

FIG. 9 illustrates an exemplary embodiment of an active sequencing complex 900 for performing a direct sequencing. The electrically-resistive barrier 901 separates the first electrolyte solution 902 from the second electrolyte solution 903. The nanopore 904 is disposed in the electrically-resistive barrier 901, and the channel of the nanopore 905 provides a path through which ions can flow between the first electrolyte 902 and the second electrolyte 903. The working electrode 906 is disposed on the side of the electrically-resistive barrier 901 containing the second electrolyte 903 (termed the “trans side” of the electrically-resistive barrier) and positioned near the nanopore 904. The counter electrode 907 is positioned on the side of the electrically-resistive barrier 901 containing the first electrolyte 902 (termed the “cis side” of the electrically-resistive barrier). The signal source 908 is adapted to apply a voltage signal between the working electrode 906 and the counter electrode 907. The first electrolyte 902 includes a nucleic acid of interest 910. The nucleic acid of interest 910 is pulled (e.g., loaded) into the nanopore by an electrical force, such as a force generated in the presence of an electric field generated by a voltage applied across the electrically-resistive barrier 901 and/or nanopore 904. As nucleotides or sequences of nucleotides occupy the channel of the nanopore 904, they affect ionic flow through the nanopore 904, thereby generating an ionic blockade signal. Different ionic blockade signals can be generated by each nucleotide or sequence of nucleotides occupying the channel 905. This process is repeated iteratively as the nucleic acid of interest 910 passes through the channel, and the sequence of the nucleic acid of interest 910 is extrapolated based on the unique sequence of ionic blockade signals recorded Feng, Manrao, and Wang I.

### IV. EXAMPLES

An a-HL pore having high conductance with a narrower ion-passage was generated by tethering a synthetic polymer inside the protein nanopore. When the nanopore captures thin and highly negatively-charged polymers, the nanopore exhibits higher conductance than it does in the absence of the captured polymer. Without being bound by theory, the mechanism of the observed enhancement in conductance may be due to the polymers carrying large counter-ion clouds. Capture of these polymers into the nanopore facilitate the transportation of these counter-ions, which result in the observed enhanced conductance state despite the narrower passage.

### A. Synthesis of a Charged Polymer

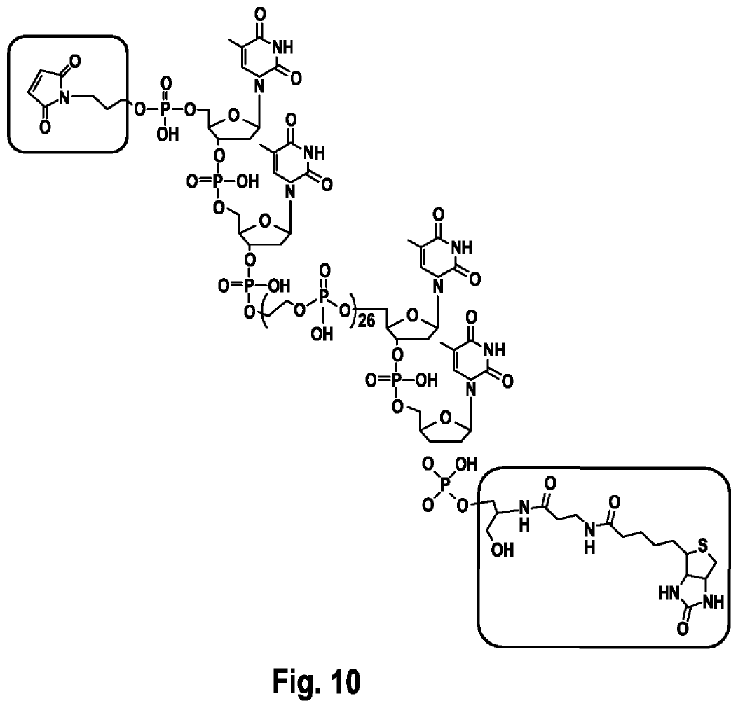

A charged polymer having the structure disclosed in FIG. 10 was synthesized on an ABI 3900 DNA Synthesizer using standard solid phase phosphoramidite chemistry protocol. The resulting polymer was cleaved from resin by concentrated ammonium hydroxide treatment. The product was concentrated on a SPEEDVAC vacuum concentrator (Thermo Fisher Scientific) and then purified by reversed-phase high-performance liquid chromatography (RP-HPLC) to give pure compound. The protected maleimide was removed by heating in dried toluene at 90° C. for three hours. The product was used without further purification. The maleimide group is highlighted in blue and the biotin group is highlighted in green.

B. Expression and purification of an αHL nanopore

Both wild-type α-HL-6×-His (SEQ ID NO: 2) and α-HL-N17C_6×-His (SEQ ID NO: 3) were expressed in BL21 DE3 Star pLys-S E. coli cells grown in MAGIC MEDIA E. coli expression media (Invitrogen) overnight at 25° C. SEQ ID NO: 3 comprises a Spy Tag near the C-terminus. Each was lysed by sonication in 25 mM Tris, pH 8.0, 300 mM NaCl, 10 mM imidazole. Both of them were purified on a TALON column and eluted in the same buffer with 150 mM imidazole. The his tag of WT α-HL-6×-His was cleaved by TEV protease to generate WT a-HL (SEQ ID NO: 1).

### C. Pore Assembly and Purification

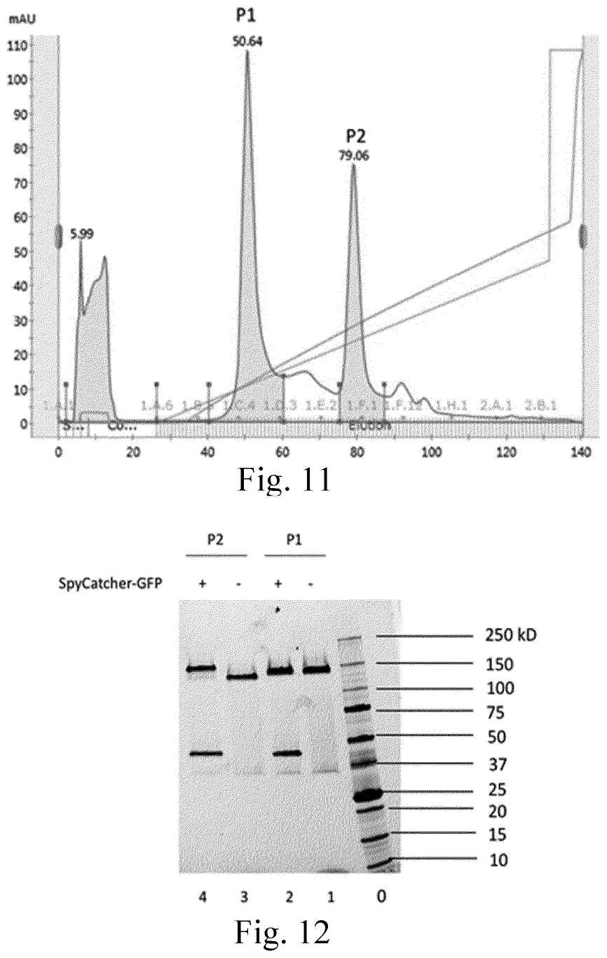

A 1:6 oligomer which has one of α-HL-N17C-SpyTag-6×-His (SEQ ID NO: 3) and six of WT a-HL (SEQ ID NO: 1), the purified α-HL-N17C-SpyTag-6×-His (G124) monomer was mixed with the his-tag cleaved WT a-HL (G1471) monomer at a ratio of 1:8 (w/w). The lipid 1,2-diphytanoyl-sn-glycero-3-phosphocholine (DPhPC) was added until the final concentration of the lipid became 5 mg/mL. The mixture was incubated overnight at 37° C. Lipid vesicles were solubilized in buffer containing 5% (w/v) n-octyl-β-D-glucoside (B-OG). Oligomers were purified by cation exchange chromatography on a RESOURCE S column in 20 mM sodium acetate (NaAc) buffer, pH 4.8, 30 mM NaCl, 0.1% Tween 20, 1 mM tris(2-carboxyethyl) phosphine (TCEP). Bound proteins were eluted with a linear gradient of 20 mM NaAc buffer, pH 4.8, 2 M NaCl, 0.1% Tween 20, 1 mM tris(2-carboxyethyl) phosphine (TCEP). As seen in FIG. 11, two major peaks were obtained, peak 1 (P1) and peak 2 (P2). Peak 1 corresponded to 0:7 oligomers and peak 2 corresponded to 1:6 oligomers.

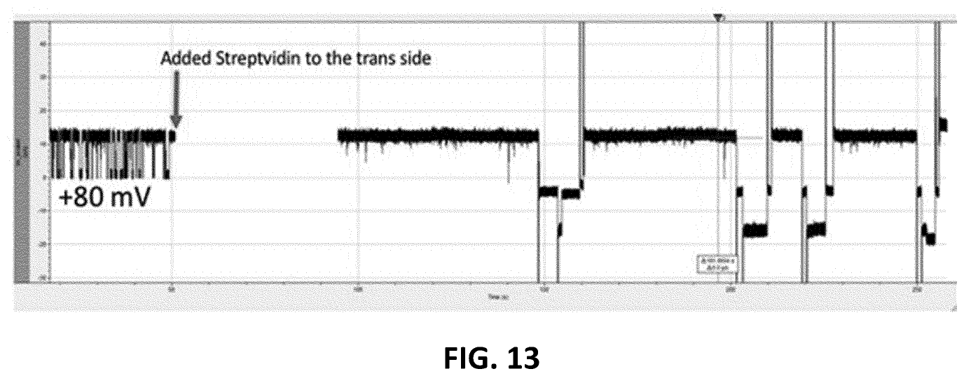

The 1:6 oligomer (P2) was confirmed by adding SpyCatcher-GFP protein and running SDS polyacrylamide gel. Results are illustrated in FIG. 12. Peak P1 does not show electrophoretic mobility shift when incubated with SpyCatcher-GFP, indicating it is a 0:7 oligomer. Peak P2 shows electrophoretic mobility shift when incubated with SpyCatcher-GFP, indicating it is a 1:6 oligomer.

D. Chemical Conjugation of the 1:6 Pore with the Charged Polymer

The 1:6 oligomer was mixed with the polymer tag at a ratio of 1:10 (mol:mol) in 20 mM HEPES pH7.5, 100 mM NaCl, 0.01% Tween 20, followed by incubation at room temperature for 4 hr. The conjugate pore was bound to MagStrep “type3” XT Beads and eluted with elution buffer (20 mM HEPES pH7.5, 100 mM NaCl, 0.001% Tween 20, 8% (w/v) Trehalose, 2 mM d-Biotin).

### E. Single Channel Recordings

A planar bilayer was formed across an aperture of ˜100 μm in a polytetrafluoroethylene film by the ‘Montal-Muller’ approach. In this approach, the aperture was first treated with a drop of 10% hexadecane/pentane. A droplet (˜ 5 uL) of 10 mg/mL DPhPC (1,2-diphytanoyl-sn-glycerophosphocholine) pentane solution was applied on the top surface of buffer solutions (200 mM KGlu, 0.5 mM EDTA, 20 mM HEPES pH 7.5) in each chamber and a lipid monolayer instantly formed as the solvent evaporated and then a bilayer was created by raising the lipid monolayers on the buffer solutions across both sides of the aperture.

A pair of Ag/AgGlu electrodes were prepared. The ground electrode was connected to the cis compartment, the working electrode to trans side. The purified 1:6 oligomer was added to the cis side. The electrical current was detected with a pair of Ag/AgGlu electrodes, amplified with a patch-clamp amplifier equipped with HUMMSILENCER technology (AXON AXOPATCH 200B microelectrode amplifier; Axon Instruments), filtered with a low-pass Bessel filter (80 dB/decade) with a corner frequency of 1 kHz and then digitized with a DIGIDATA 1200 A/D converter (Axon Instruments) at a sampling frequency of 5 kHz. Data samples were stored on the hard disk of a PC computer.

A first set of capture events were recorded with the charged polymer tethered only at the first end. Then, streptavidin was flowed onto the trans side of the bilayer and an additional set of recordings were then generated. Results are shown at FIG. 13. Fluctuating currents were observed with two levels, which implies that the tethered polymer occupies two states: 1) inserted into the channel and 2) ejected from the channel. Addition of streptavidin to the trans side led to higher conductance between the two levels. This is indicative of the biotin at the terminus of the polymer becoming anchored by the streptavidin, thus causing the polymer to remain in a permanently threaded position (FIG. 4).

Conductances were then measured for the charged polymer conjugated/streptavidin captured nanopore at a variety of voltages and compared to the same pore without the charged polymer. Results are shown at FIG. 2. As can be seen, the charged polymer-conjugated pore (line with square hatch marks) consistently had larger conductance levels than the same pore without the charged polymer.

### V. REFERENCES

1. Ayub et al., Nucleobase Recognition by Truncated α-Hemolysin Pores, ACS Nano, 2015, Vol. 9, Issue 8, 7895-7903.
2. Chen & Liu, Fabrication and Applications of Solid-State Nanopores, 2019, Sensors, Vol. 19, Issue 8, E1886.
3. Feng et al., Nanopore-based Fourth-generation DNA Sequencing Technology, 2015, Genomics, Proteomics & Bioinformatics, Vol. 13, Issue 1, pp. 4-16.
4. Gari et al., Quiet Outer Membrane Protein G (OmpG) Nanopore for Biosensing, ACS Sensors, Apr. 14, 2019, Vol. 4, pp. 1230-35.
5. Hammerstein et al., Subunit dimers of α-hemolysin expand the engineering toolbox for protein Nanopores, Journal of Biological Chemistry, Vol. 286, Issue 16, pp. 14324-34.
6. Haque et al., Solid-State and Biological Nanopore for Real-Time Sensing of Single Chemical and Sequencing of DNA, Nano Today (February 2013), Vol. 8, Issue 1, pp. 56-74.
7. Kumar et al., PEG-labeled nucleotides and nanopore detection for single molecule DNA sequencing by synthesis, 2012, Scientific Reports, Vol. 2, Art. 684.
8. Lee et al., Recent progress in solid-state nanopores, 2018, Advanced Materials, Vol. 30, Issue 42.
9. Manrao et al., Reading DNA at single-nucleotide resolution with a mutant MspA nanopore and phi29 DNA polymerase, 2012, Nature Biotechnology, Vol. 30, pp. 349-53.
10. Pavlenok & Niederweis, Hetero-oligomeric MspA pores in Mycobacterium smegmatis, 2016, FEMS Microbiology Letters, Vol. 363, Issue 7, fnw046.
11. Wang et al., The evolution of nanopore sequencing, 2015, Frontiers in Genetics, Vol. 5, Art. 449 (“Wang I”).
12. Wang et al., Engineering of Protein Nanopores for Sequencing, Chemical or Protein Sensing and Disease Diagnosis, 2018, Current Opinions in Biotechnology, Vol. 51, pp. 80-89 (“Wang II”).
13. Wasfi et al., Graphene-based nanopore approaches for DNA sequencing: A literature review, 2018, Biosensors and Bioelectronics, Vol. 119, pp. 191-203.

1. A nanopore-forming protein comprising:
a channel having an entrance side and an exit side,
a charged polymer threaded through the channel, comprising:
a first end fixed in place on the entrance side of the channel,
a second end disposed on the exit side of the channel, optionally fixed in place, and
a negatively charged region disposed between the first and second ends and extending substantially the entire length of the channel.

2. The nanopore of claim 1, wherein the channel is formed by 7 monomer subunits, each monomer subunit having at least 75% sequence identity to SEQ ID NO: 1, and wherein the first end is covalently bound to one of the 7 monomer subunits.

3. The nanopore of claim 1, wherein the second end of the charged polymer comprises biotin or a biotin derivative and wherein the biotin or biotin derivative is fixed to avidin, streptavidin, or deglycosylated avidin disposed on the exit side of the channel.

4. The nanopore of claim 1, wherein the second end of the charged polymer comprises an antibody epitope and wherein the antibody epitope is fixed to an antibody disposed on the exit side of the channel.

5. The nanopore of claim 1, wherein the negatively charged region comprises at least 10 phosphodiester bonds.

6. The nanopore of claim 5, wherein the phosphodiester bonds link nucleotides and/or abasic sites.

7. The nanopore of claim 6, wherein the negatively charged region comprises at least 10 abasic sites.

8. The nanopore of claim 6, wherein the negatively charged region comprises at least 20 abasic sites.

9. The nanopore of claim 6, wherein the abasic site has the following structure: 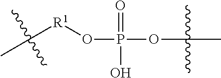
wherein R¹ is an alkyl chain from 2 to 10 carbons in length.

10. The nanopore of claim 6, wherein the charged polymer has the following structure: 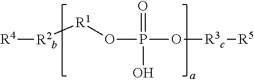
wherein:
a is from 10 to 100,
R¹ is an alkyl chain from 2 to 10 carbons in length,
R² and R³ are nucleotides,
b is from 0 to 10
c is from 0 to 10
one of R⁴ and R⁵ is the first end, and
the other of R⁴ and R⁵ is the second end.

11. The nanopore of claim 10, wherein a nucleobase of both R² and R³ is a pyrimidine or pyrimidine derivative.

12. The nanopore of claim 2, wherein at least 6 of the 7 subunits comprise D111, M113, and K147 when aligned with SEQ ID NO: 1.

13. A system for performing nanopore-based sequencing, the system comprising a chip comprising a plurality of nanopore sequencing complexes and a computing system adapted to record changes in one or more electrical characteristics of the nanopore sequencing complexes, wherein each nanopore sequencing complex comprises:
(a) an electrochemically resistive barrier disposed on a surface of the chip, wherein the barrier has a cis side and a trans side;
(b) a first electrolyte solution on the cis side of the barrier;
(c) a second electrolyte solution on the trans side of the barrier; and
(d) a nanopore according to claim 1, wherein the entrance side of the channel is on the cis side of the barrier and the exit side of the channel is on the trans side of the barrier, such that the channel permits ion exchange between the first electrolyte solution and the second electrolyte solution, and
(e) at least one electrode in electronic communication with the computing system, wherein the electrode is positioned to detect changes in at least one electrical characteristic of the nanopore sequencing complex associated with occupation of the nanopore by a molecule and to transmit the detected change(s) to the computing system.

14. A method of sequencing a template nucleic acid on a system according to claim 13, the method comprising:
(a) generating a plurality of active nanopore sequencing complexes, each active nanopore sequencing complex comprising a single stranded nucleic acid template inserted into the channel of the nanopore;
(b) at each active sequencing complex, applying a force to the single stranded nucleic acid template, wherein the force causes the single stranded nucleic acid to move through the channel from the entrance side to the exit side, and wherein each nucleotide or sequence of nucleotides of the nucleic acid causes a unique change in an electrical characteristic of the nanopore;
(c) detecting the change in the electrical characteristic of the nanopore caused by the nucleotide or sequence of nucleotides occupying the channel and recording the change on the computer system; and
(d) correlating each recorded change to the nucleotide or sequence of nucleotides occupying the channel, thereby generating a sequence of the single stranded template nucleic acid at that electrode.

15. A system for performing Sequencing-by-Synthesis (SBS) nucleic acid sequencing, the system comprising a chip comprising a plurality of nanopore sequencing complexes and a computing system adapted to record changes in one or more electrical characteristics of the nanopore sequencing complexes, wherein each nanopore sequencing complex comprises:
(a) an electrochemically resistive barrier disposed on a surface of the chip, wherein the barrier has a cis side and a trans side;
(b) a first electrolyte solution on the cis side of the barrier;
(c) a second electrolyte solution on the trans side of the barrier; and
(d) a nanopore according to claim 1, wherein the entrance side of the channel is on the cis side of the barrier and the exit side of the channel is on the trans side of the barrier, such that the channel permits ion exchange between the first electrolyte solution and the second electrolyte solution;
(e) at least one electrode in electronic communication with the computing system, wherein the electrode is positioned to detect changes in at least one electrical characteristic of the nanopore sequencing complex associated with occupation of the nanopore by a molecule and to transmit the detected change(s) to the computing system;
(f) a nucleic acid polymerase associated with the nanopore on the cis side of the barrier; and
(g) a set of polymer tagged nucleoside-5 [symbol] -oligophosphates (N5OP) disposed in the first electrolyte solution.

16. A method of sequencing a template nucleic acid on a system according to claim 15, the method comprising:
(a) generating a plurality of active nanopore sequencing complexes, each active nanopore sequencing complex comprising:
(b) a single stranded nucleic acid template complexed with the nucleic acid polymerase;
(c) a primer hybridized to the template nucleic acid; and
(d) at least one of the set of tagged N5OP associated with the polymerase,
(e) at each active sequencing complex, iteratively linking the tagged N5OP to the primer by a template-dependent nucleic acid amplification reaction catalyzed by the nucleic acid polymerase, wherein the polymer tag of the tagged N5OP moves into or in proximity to the channel of the nanopore as the tagged N5OPs is linked to the complementary nucleic acid, and wherein movement of the polymer tag into or in proximity to the channel changes the electrical characteristic of the nanopore;
(f) detecting the change in the electrical characteristic of the nanopore caused by the polymer tags and recording the change on the computer system; and
(g) correlating each recorded change to one of the tagged N5OPs, thereby generating a sequence of the complementary nucleic acid generated at that electrode.
